(19) United States
(12) Patent Application Publication
(10) Pub. No.: US 2018/0108845 A1
(43) Pub. Date: Apr. 19, 2018

(54) CYCLIC UREA COMPOUNDS FOR ELECTRONIC DEVICES

(71) Applicants: Dow Global Technologies LLC, Midland, MI (US); Rohm and Haas Electronic Materials Korea Ltd., Cheonan (KR)

(72) Inventors: MARK E. Ondari, Midland, MI (US); Robert J. Wright, Sugar Land, TX (US); Robert DJ Froese, Midland, MI (US); David D. Devore, Midland, MI (US); Hong-Yeop Na, Seoul (KR)

(21) Appl. No.: 15/567,617

(22) PCT Filed: Jun. 28, 2016
(86) PCT No.: PCT/US2016/039768

§ 371 Date: Oct. 19, 2017

Related U.S. Application Data
(60) Provisional application No. 62/186,475, filed on Jun. 30, 2015.

(51) Int. Cl. H01L 51/00; C07D 233/36

(52) U.S. Cl. CPC H01L 51/0061; H01L 51/5056; H01L 51/0067; C07D 233/36

(57) ABSTRACT

A composition is provided, which comprises at least one cyclic urea compound of Formula 1, as described herein. The composition can be used in electronic devices, such as organic electroluminescent devices.

### REFERENCE TO RELATED APPLICATIONS

The present application claims the benefit of U.S. Provisional Application No. 62/186,475, filed on Jun. 30, 2015, and incorporated herein by reference.

### BACKGROUND

Organic electroluminescence (EL) devices are display devices that employ stacks of films containing organic aromatic compounds as an electroluminescent layer. Such compounds are generally classified as electroluminescent materials and charge transport materials. Several properties required for such electroluminescent and charge transport compounds include high fluorescent quantum yield in solid state, high mobility of electrons and holes, chemical stability during vapor-deposition in vacuum, and the ability to form stable films. These desired features increase the lifetime of an EL device. There is a continual need for improved electroluminescent compounds and films containing the same.

JP2011136910A (abstract) describes cyclic diimides (piperazine-2,5-dione)-based bis(triarylamine) “derivative for organic electroluminescent element, organic photoconducting materials, photoelectric conversion element, solar cell, image sensor and hole injection materials.” Also U.S. Pat. No. 8,022,617 B2 describes a charge transport material represented by the following formula:

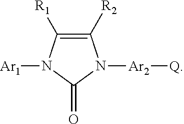

However, such compounds have HOMO values that are deeper than the desirable range useful for improved organic electroluminescent device.

There is a continued need to provide compounds which can prepare an organic electroluminescent device having improvements on performance, and an organic electroluminescent device comprising the same. These needs have been met by the following invention, where compounds of the following composition are introduced.

### SUMMARY OF INVENTION

Provided is a composition comprising at least one compound of Formula 1 below:

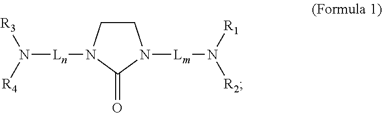

wherein, R1, R2, R3 and R4 are each, independently, selected from the following: an unsubstituted alkyl, a substituted alkyl, an unsubstituted heteroalkyl, a substituted heteroalkyl, an unsubstituted aryl, a substituted aryl, an unsubstituted heteroaryl, or a substituted heteroaryl; and wherein R1 and R2 may be optionally fused to form one or more ring structures; and wherein R3 and R4 may be optionally fused to form one or more ring structures; and

Ln and Lm are each, independently, selected from the following: an unsubstituted alkylene, a substituted alkylene, an unsubstituted heteroalkylene, a substituted heteroalkylene, an unsubstituted arylene, a substituted arylene, an unsubstituted heteroarylene, or a substituted heteroarylene; and

wherein, for Formula 1, one or more hydrogen atoms may optionally be substituted with deuterium.

### DETAIL DESCRIPTION

As discussed above, a composition is provided, which comprises at least one compound of Formula 1 below:

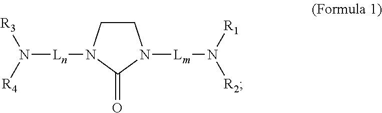

wherein, R1, R2, R3 and R4 are each, independently, selected from the following: an unsubstituted alkyl, a substituted alkyl, an unsubstituted heteroalkyl, a substituted hetero-alkyl, an unsubstituted aryl, a substituted aryl, an unsubstituted heteroaryl, or a substituted heteroaryl; and wherein R1 and R2 may be optionally fused to form one or more ring structures; and wherein R3 and R4 may be optionally fused to form one or more ring structures; and

Ln and Lm are each, independently, selected from the following: an unsubstituted alkylene, a substituted alkylene, an unsubstituted heteroalkylene, a substituted hetero-alkylene, an unsubstituted arylene, a substituted arylene, an unsubstituted heteroarylene, or a substituted heteroarylene; and

wherein, for Formula 1, one or more hydrogen atoms may optionally be substituted with deuterium.

An inventive composition may have a combination of two or more embodiments described herein.

The “at least one compound of Formula 1” may have a combination of two or more embodiments as described herein.

As used herein, for each Formula 1-4, R1=R₁, R2=R₂, and so on.

In one embodiment, for Formula 1, Lₘ and Lₙ each independently, an unsubstituted (3- to 30-membered)heteroarylene, a substituted (3- to 30-membered)hetero-arylene, an unsubstituted (C6-C30)arylene, or a substituted (C60C30)arylene.

In one embodiment, for Formula 1, Lₘ and Lₙ each independently, selected from one of the following structures:

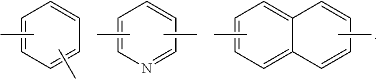

In one embodiment, for Formula 1, R1, R2, R3 and R4, are each, independently, selected from an unsubstituted (C6-C30)arylene, or a substituted (C6-C30)aryl, an unsubstituted (3- to 30-membered)heteroaryl, or a substituted (3- to 30-membered)heteroaryl.

In one embodiment, R₁=R₃ and R₂=R₄.

In one embodiment, R₁=R₄ and R₂=R₃.

In one embodiment, R₁=R2=R3=R₄.

In one embodiment, for Formula 1, R₁, R₂, R₃ and R₄, are each, independently, selected from the following A1 to A48:

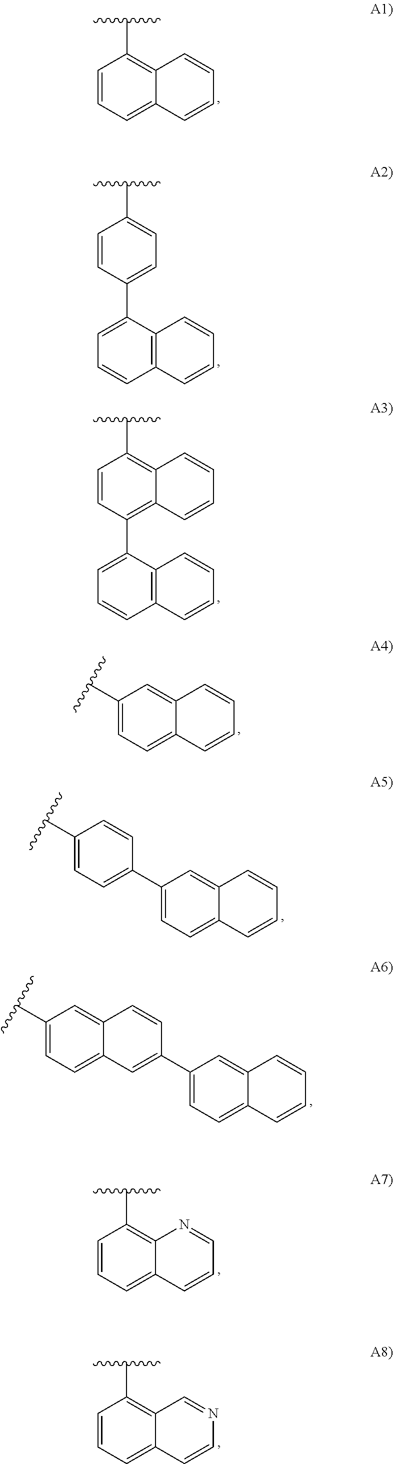

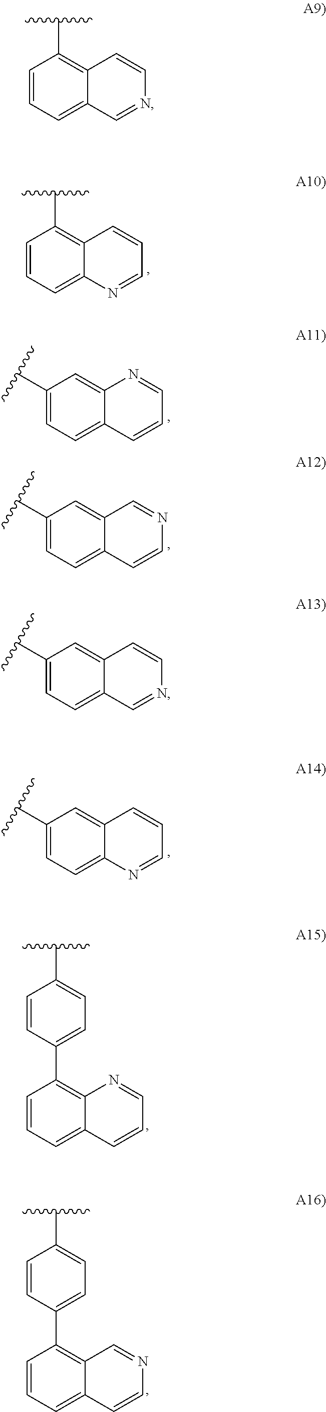

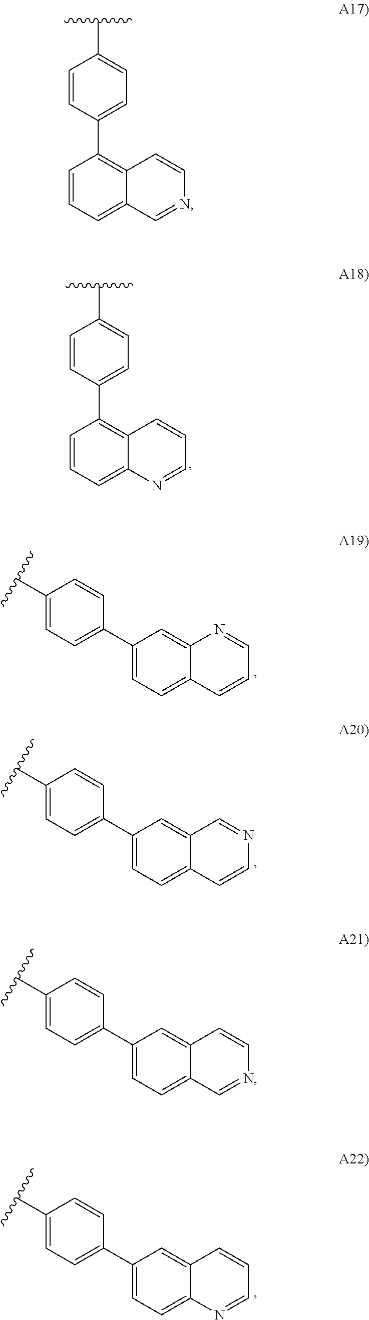 [SMILES: *c1ccc(-c2cccc3cnccc23)cc1 ; *c1ccc(-c2cccc3ncccc23)cc1 ; *c1ccc(-c2ccc3cccnc3c2)cc1 ; *c1ccc(-c2ccc3ccncc3c2)cc1 ; *c1ccc(-c2ccc3cnccc3c2)cc1 ; *c1ccc(-c2ccc3ncccc3c2)cc1]

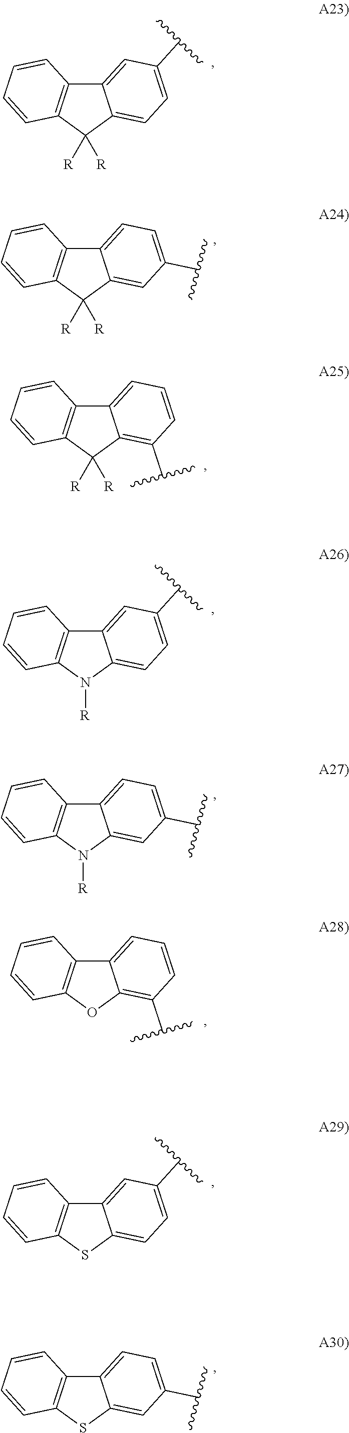

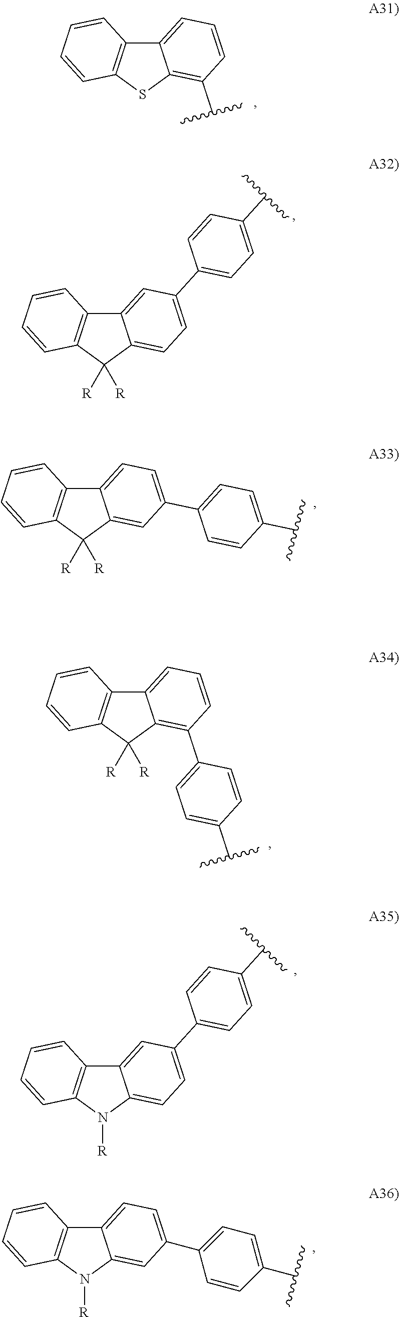

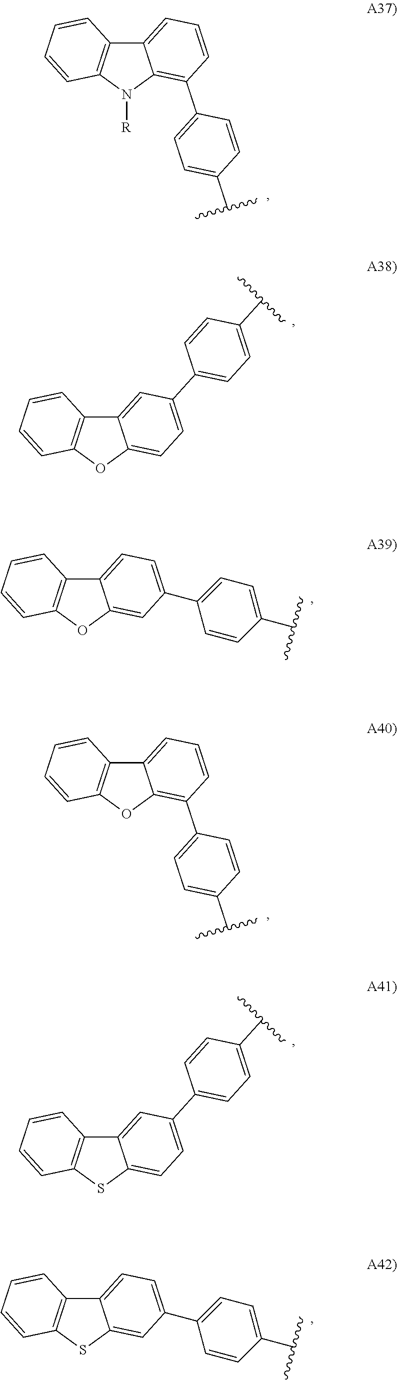

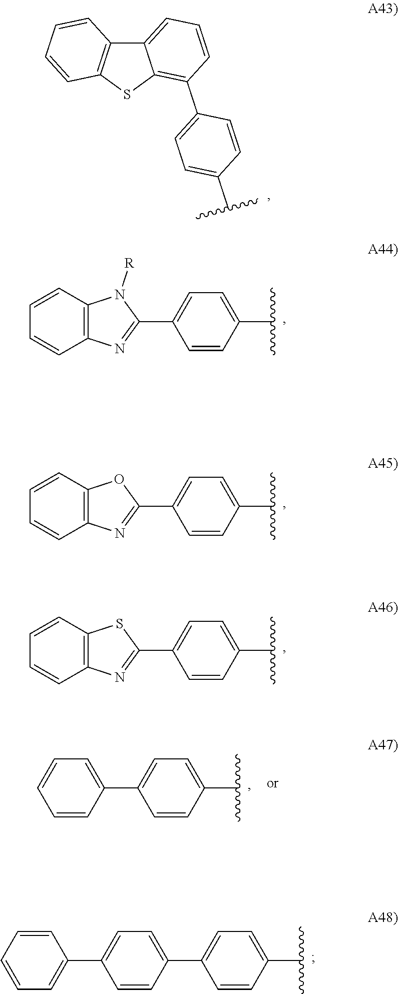

and
wherein for structures A24) through A27), A32) through A38), and A44) each R is independently an alkyl. In one embodiment, for Formula 1, R₁, R₂, R₃ and R₄, are each, independently, selected from the following: A1) through A6), A32) through A37), A47) and A48). In one embodiment, for Formula 1, R₁, R₂, R₃ and R₄, are each, independently, selected from the following: A1) through A6), A47) and A48).

For the above structures and other structures noted herein, the external connection point of each substituent is indicated by a wavy line, as recommended by current IUPAC standards: Pure Appl. Chem., 2008, 80, 277 (Graphical representation standards for chemical structural diagrams).

In one embodiment, Formula 1 is selected from Formula 1a:

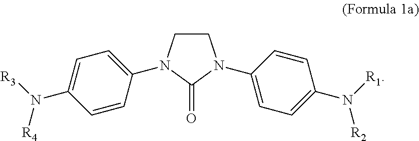

In one embodiment, Formula 1 is selected from the following (a) through (o):

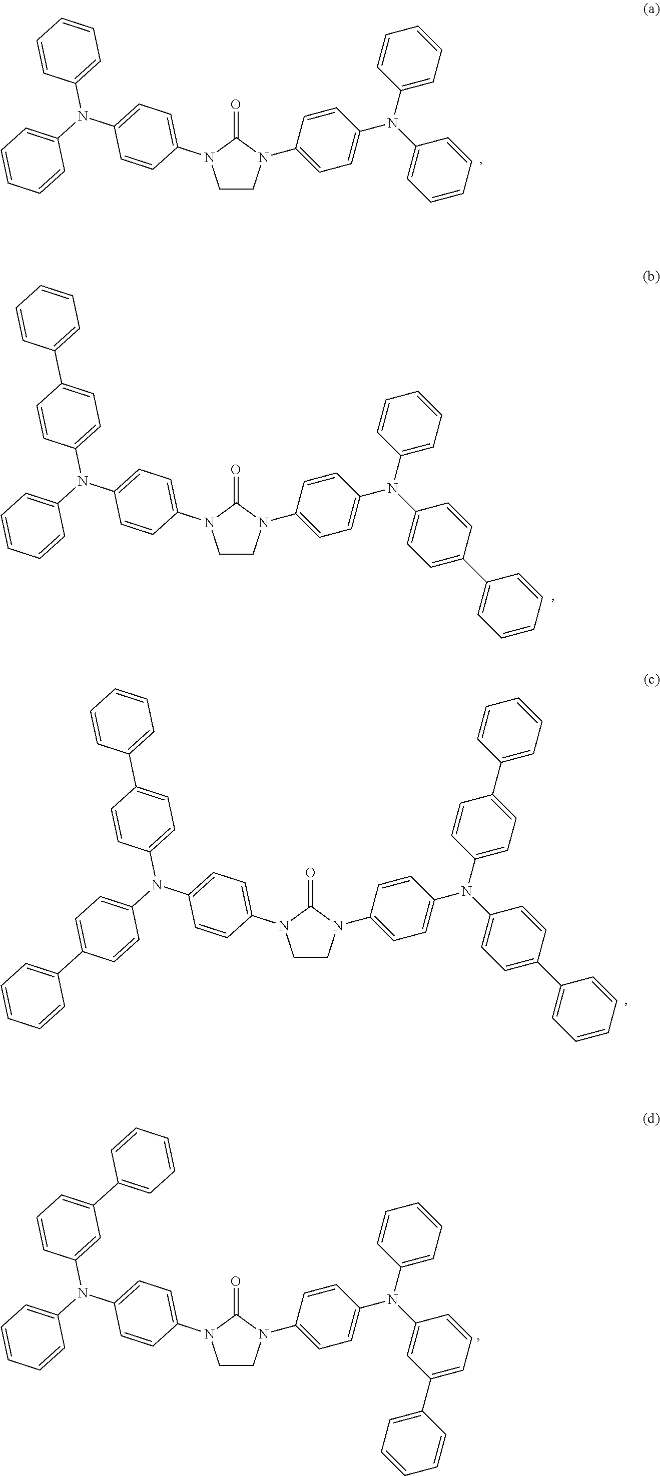 [SMILES: O=C1N(c2ccc(N(c3ccccc3)c3ccccc3)cc2)CCN1c1ccc(N(c2ccccc2)c2ccccc2)cc1 ; O=C1N(c2ccc(N(c3ccccc3)c3ccc(-c4ccccc4)cc3)cc2)CCN1c1ccc(N(c2ccccc2)c2ccc(-c3ccccc3)cc2)cc1 ; O=C1N(c2ccc(N(c3ccc(-c4ccccc4)cc3)c3ccc(-c4ccccc4)cc3)cc2)CCN1c1ccc(N(c2ccc(-c3ccccc3)cc2)c2ccc(-c3ccccc3)cc2)cc1 ; O=C1N(c2ccc(N(c3ccccc3)c3cccc(-c4ccccc4)c3)cc2)CCN1c1ccc(N(c2ccccc2)c2cccc(-c3ccccc3)c2)cc1]

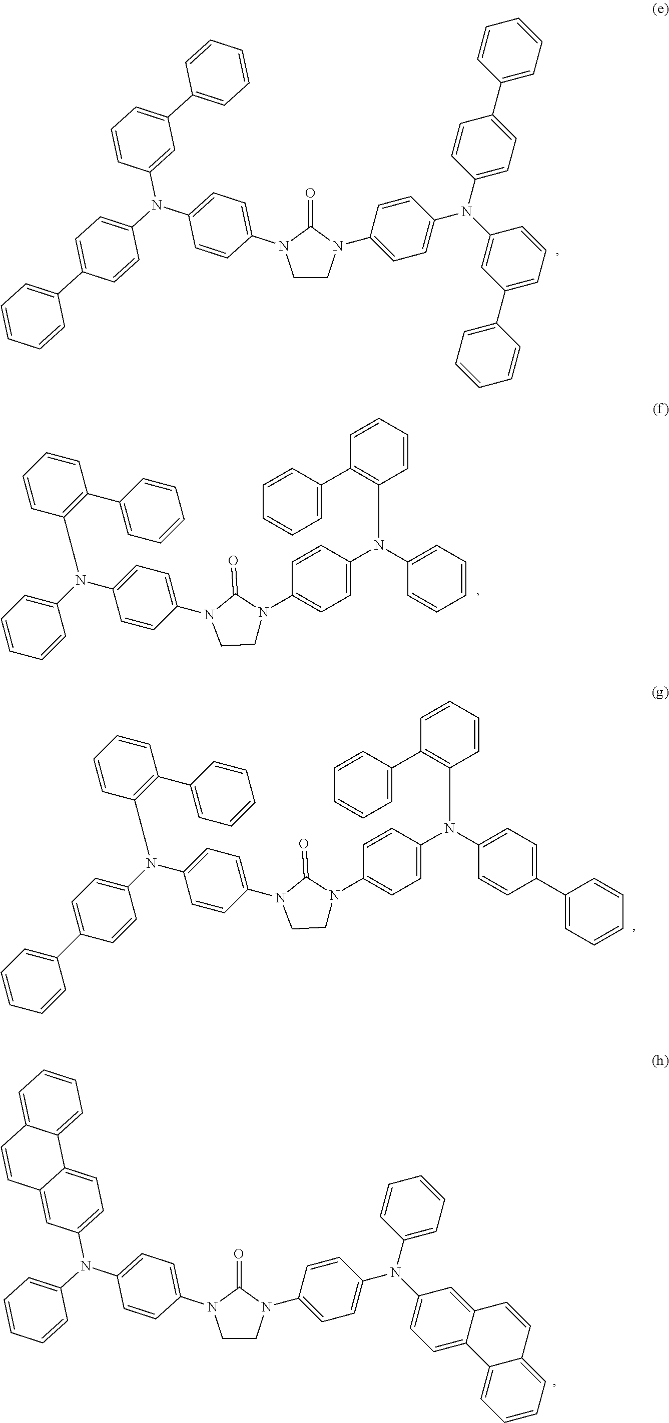 [SMILES: O=C1N(c2ccc(N(c3ccc(-c4ccccc4)cc3)c3cccc(-c4ccccc4)c3)cc2)CCN1c1ccc(N(c2ccc(-c3ccccc3)cc2)c2cccc(-c3ccccc3)c2)cc1 ; O=C1N(c2ccc(N(c3ccccc3)c3ccccc3-c3ccccc3)cc2)CCN1c1ccc(N(c2ccccc2)c2ccccc2-c2ccccc2)cc1 ; O=C1N(c2ccc(N(c3ccc(-c4ccccc4)cc3)c3ccccc3-c3ccccc3)cc2)CCN1c1ccc(N(c2ccc(-c3ccccc3)cc2)c2ccccc2-c2ccccc2)cc1 ; O=C1N(c2ccc(N(c3ccccc3)c3ccc4c(ccc5ccccc54)c3)cc2)CCN1c1ccc(N(c2ccccc2)c2ccc3c(ccc4ccccc43)c2)cc1]

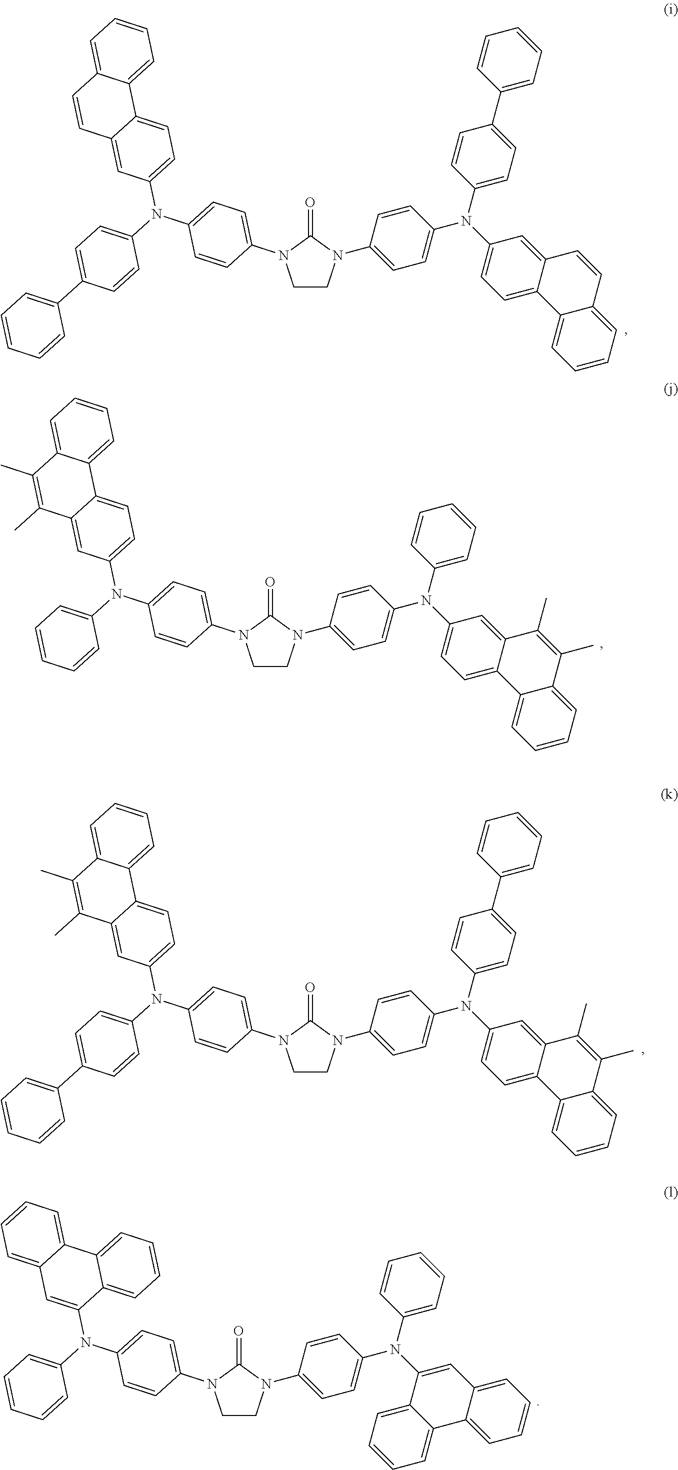

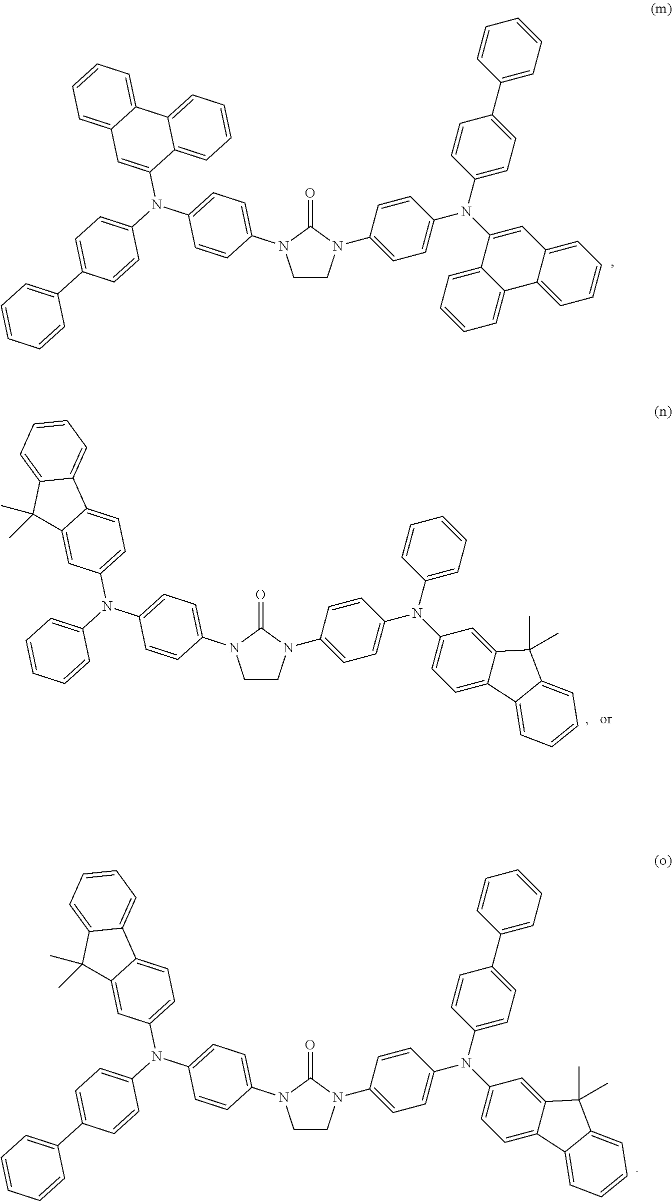 [SMILES: O=C1N(c2ccc(N(c3ccc(-c4ccccc4)cc3)c3cc4ccccc4c4ccccc34)cc2)CCN1c1ccc(N(c2ccc(-c3ccccc3)cc2)c2cc3ccccc3c3ccccc23)cc1 ; CC1(C)c2ccccc2-c2ccc(N(c3ccccc3)c3ccc(N4CCN(c5ccc(N(c6ccccc6)c6ccc7c(c6)C(C)(C)c6ccccc6-7)cc5)C4=O)cc3)cc21 ; CC1(C)c2ccccc2-c2ccc(N(c3ccc(-c4ccccc4)cc3)c3ccc(N4CCN(c5ccc(N(c6ccc(-c7ccccc7)cc6)c6ccc7c(c6)C(C)(C)c6ccccc6-7)cc5)C4=O)cc3)cc21]

In one embodiment, Formula 1 is selected from (a) through (i), (m), (n) or (o).

In one embodiment, Formula 1 is selected from (a) through (e), (n) or (o).

In one embodiment, Formula 1 is selected from (a) through (e).

In one embodiment, the compound of Formula 1 has a molecular weight greater than, or equal to, 450 g/mole.

In one embodiment, the compound of Formula 1 has a molecular weight from 450 to 1000 g/mole, or from 450 to 900 g/mole, or from 450 to 800 g/mole.

In one embodiment, the compound of Formula 1 has a HOMO level from −4.60 to −4.75 eV, or from −4.60 to −4.70 eV.

In one embodiment, the compound of Formula 1 has a LUMO level from −0.90 to −0.10 eV, or from −0.90 to −0.20 eV, or from −0.90 to −0.30 eV, or from −0.90 to −0.40 eV.

In one embodiment, the compound of Formula 1 has a glass transition temperature (Tg) from 105° C. to 170° C.

The compound of Formula 1 may have a combination of two or more embodiments described herein.

The compound of the present invention can be prepared by synthetic methods known to one skilled in the art, such as oxidative cyclization, Suzuki coupling, Hartwig-Buchwald coupling, among others.

In one embodiment, the composition comprises at least two compounds of Formula 1.

In one embodiment, the composition comprises at least three compounds of Formula 1.

In one embodiment, the composition comprises from 5 to 100 weight percent, further 10 to 99 weight percent, and further 10 to 90 weight percent, of at least one compound of Formula 1, based on the weight of the composition.

In one embodiment, the composition comprises from 50 to 90 weight percent of the compound of Formula 1, based on the weight of the composition. In a further embodiment, the composition comprises from 50 to 80 weight percent of the compound of Formula 1, based on the weight of the composition.

Also is provided is an article comprising at least one component formed from an inventive composition. In a further embodiment, the article is an organic electro-luminescent device.

Also is provided is an article comprising at least one component formed from the composition of any one embodiment, or a combination of two or more embodiments, described herein. In one embodiment, the article is an organic electroluminescent device.

Also is provided is a film comprising at least one layer formed from an inventive composition of any one embodiment, or a combination of two or more embodiments, described herein.

Also is provided an electronic device comprising at least one component formed from an inventive composition of any one embodiment, or a combination of two or more embodiments, described herein.

An inventive composition may have a combination of two or more embodiments described herein.

An inventive article may have a combination of two or more embodiments described herein.

An inventive film may have a combination of two or more embodiments described herein.

An inventive electronic device may have a combination of two or more embodiments described herein.

The organic electroluminescent device comprises a first electrode; a second electrode; and an organic layer between the first electrode and the second electrode. The organic layer comprises the compound of the present invention or a combination of the compound of the present invention and a reductive dopant.

The first electrode is formed on a substrate. The first electrode may be an anode or a cathode. The organic layer is formed on the first electrode. The organic layer may comprise a light-emitting layer. In addition to the light-emitting layer, the organic layer may further comprise an electron transport layer. In addition to the light-emitting layer, the organic layer may further comprise at least one layer selected from a hole injection layer, a hole transport layer, an electron injection layer, an electron transport layer, an interlayer, a hole blocking layer, and an electron blocking layer. For example, the organic layer may comprise a light-emitting layer, an electron transport layer, and at least one selected from a hole injection layer, a hole transport layer, an electron injection layer, an interlayer, a hole blocking layer, and an electron blocking layer.

The light-emitting layer can be formed on the first electrode. The light-emitting layer can be formed by using a host material and a dopant material. The host material may be a fluorescent host material or a phosphorescent host material. The dopant material may be a fluorescent dopant material or a phosphorescent dopant material. The organic electroluminescent device of the present invention may comprise two or more light-emitting layers.

The electron transport layer can be formed between the light-emitting layer and the second electrode or between the first electrode and the light-emitting layer. The organic electroluminescent device of the present invention may comprise two or more electron transport layers. Some known electron transport compound includes, for example, oxazole-based compounds, isoxazole-based compounds, triazole-based compounds, isothiazole-based compounds, oxadiazole-based compounds, thiadiazole-based compounds, perylene-based compounds, anthracene-based compounds, aluminum complexes, and gallium complexes.

The second electrode is formed on the organic layer. The second electrode may be an anode or a cathode.

For the organic electroluminescent device of the present invention, each of the layers such as electrodes can be formed by a technique(s) which was known in the field, and includes, for example, vacuum evaporation, sputtering, wet film-forming methods and a laser induced thermal imaging method.

### Definitions

The term “hydrocarbon,” as used herein, refers to a chemical group containing only hydrogen and carbon atoms.

The term “substituted hydrocarbon,” as used herein, refers to a hydrocarbon, in which at least one hydrogen atom is substituted with a heteroatom or a chemical group containing at least one heteroatom. Heteroatoms include, but are not limited to, 0, N, P and S.

The term “aryl,” as described herein, refers to an organic radical derived from aromatic hydrocarbon by deleting one hydrogen atom therefrom. An aryl group may be a monocyclic and/or fused ring system, each ring of which suitably contains from 4 to 7, preferably from 5 or 6 atoms. Structures wherein two or more aryl groups are combined through single bond(s) are also included. Specific examples include, but are not limited to, phenyl, naphthyl, biphenyl, anthryl, indenyl, fluorenyl, benzofluorenyl, phenanthryl, triphenylenyl, pyrenyl, perylenyl, chrysenyl, naphtacenyl, fluoranthenyl and the like, but are not restricted thereto. The naphthyl may be 1-naphthyl or 2-naphthyl, the anthryl may be 1-anthryl, 2-anthryl or 9-anthryl, and the fluorenyl may be any one of 1-fluorenyl, 2-fluorenyl, 3-fluorenyl, 4-fluorenyl and 9-fluorenyl. In one embodiment, the aryl is selected from phenyl, naphthyl, biphenyl, anthryl, indenyl, fluorenyl, benzofluorenyl, phenanthryl, triphenylenyl, pyrenyl, perylenyl, chrysenyl, naphtacenyl, or fluoranthenyl.

The term “substituted aryl,” as used herein, refers to an aryl, in which at least one hydrogen atom is substituted with a heteroatom or a chemical group containing at least one heteroatom. Heteroatoms include, but are not limited to, 0, N, P and S.

The term “heteroaryl,” as described herein, refers to an aryl group, in which at least one carbon atom or CH group or CH₂ is substituted with a heteroatom (for example, B, N, O, S, P(═O), Si and P) or a chemical group containing at least one heteroatom (for example, —N(R)—). The heteroaryl may be a 5- or 6-membered monocyclic heteroaryl or a polycyclic heteroaryl which is fused with one or more benzene ring(s), and may be partially saturated. The structures having one or more heteroaryl group(s) bonded through a single bond are also included. The heteroaryl groups may include divalent aryl groups of which the heteroatoms are oxidized or quarternized to form N-oxides, quaternary salts, or the like. Specific examples include, but are not limited to, monocyclic heteroaryl groups, such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl; polycyclic heteroaryl groups, such as benzofuranyl, fluoreno[4, 3-b]benzo-furanyl, benzothiophenyl, fluoreno[4, 3-b]benzothiophenyl, isobenzofuranyl, benzimidazolyl, benzothiazolyl, benzisothiazolyl, benzisoxazolyl, benzoxazolyl, isoindolyl, indolyl, indazolyl, benzothia-diazolyl, quinolyl, isoquinolyl, cinnolinyl, quinazolinyl, quinoxalinyl, carbazolyl, phenanthridinyl and benzodioxolyl; and corresponding N-oxides (for example, pyridyl N-oxide, quinolyl N-oxide) and quaternary salts thereof. In one embodiment, the heteroaryl is selected from furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl; benzofuranyl, fluoreno[4, 3-b]benzo-furanyl, benzothiophenyl, fluoreno[4, 3-b]benzothiophenyl, isobenzofuranyl, benzimidazolyl, benzothiazolyl, benzisothiazolyl, benzisoxazolyl, benzoxazolyl, isoindolyl, indolyl, indazolyl, benzothia-diazolyl, quinolyl, isoquinolyl, cinnolinyl, quinazolinyl, quinoxalinyl, carbazolyl, phenanthridinyl or benzodioxolyl.

The term “substituted heteroaryl,” as used herein, refers to a heteroaryl, in which at least one hydrogen atom is substituted with a heteroatom or a chemical group containing at least one heteroatom. Heteroatoms include, but are not limited to, 0, N, P and S.

The term “alkyl,” as described herein, refers to an organic radical derived from an aliphatic hydrocarbon by deleting one hydrogen atom therefrom. An alkyl group may be a linear, branched and/or cyclic. Specific examples include, but are not limited to, methyl, ethyl, propyl, cyclohexyl, cylcopentyl.

The term “substituted alkyl,” as used herein, refers to an alkyl, in which at least one hydrogen atom is substituted with a heteroatom or a chemical group containing at least one heteroatom. Heteroatoms include, but are not limited to, O, N, P and S.

The term “heteroalkyl,” as described herein, refers to an alkyl group, in which at least one carbon atom or CH group or CH₂ is substituted with a heteroatom (for example, B, N, O, S, P(═O), Si and P) or a chemical group containing at least one heteroatom (for example, —N(R)—).

The term “substituted heteroalkyl,” as used herein, refers to a heteroaryl in which at least one hydrogen atom is substituted with a heteroatom or a chemical group containing at least one heteroatom. Heteroatoms include, but are not limited to, O, N, P and S.

### EXPERIMENTAL

### Reagents and Test Methods

All solvents and reagents were obtained from commercial vendors, including Sigma-Aldrich, Fisher Scientific, Acros, TCI and Alfa Aesar, and were used in the highest available purities, and/or were, when necessary, recrystallized before use. Dry solvents were obtained from in-house purification/dispensing system (hexane, toluene, tetrahydrofuran and diethyl ether), or purchased from Sigma-Aldrich. All experiments involving water sensitive compounds were conducted in “oven dried” glassware, under nitrogen atmosphere, or in a glovebox. Reactions were monitored by analytical thin-layer chromatography (TLC) on precoated aluminum plates (VWR 60 F254), visualized by UV light and/or potassium permanganate staining. Flash chromatography was performed on an ISCO COMBIFLASH system with GRACERESOLV cartridges.

1H-NMR-spectra (500 MHz or 400 MHz) were obtained on a Varian VNMRS-500 or VNMRS-400 spectrometer at 30° C., unless otherwise noted. The chemical shifts were referenced, depending on the NMR solvent used, to one of the following: TMS in CHCl₃ (δ=0.00) in CDCl3, Benzene-d₅ (7.15) in Benzene-d₆ or DMSO-d₅ (δ 2.50) in DMSO-d₆. If necessary, peak assignment was carried out with the help of COSY, HSQC or NOESY experiments to confirm structural identity.

¹³C-NMR spectra (125 MHz or 100 MHz) were obtained on a Varian VNMRS-500 or VNRMS-400 spectrometer, and referenced, depending on the NMR solvent used, to solvent or standard signals (0.0—TMS in CDCl₃, 128.02—Benzene-d₆, 39.43—DMSO-d₆).

Routine LC/MS studies were carried out as follows. Five microliter aliquots of the sample, as “3 mg/ml solution in THF,” were injected on an AGILENT 1200SL binary gradient liquid chromatography, coupled to an AGILENT 6520 QTof, quadrupole-time of flight MS system, via a dual spray electrospray (ESI) interface operating in the PI mode. The following analysis conditions were used: column: 150×4.6 mm ID, 3.5 μm ZORBAX SB-C8; column temperature: 40° C.; mobile phase: 75/25 A/B to 15/85 A/B at 40 minutes; solvent A=0.1v % formic acid in water; solvent B=THF; flow 1.0 mL/min; UV detection: diode array 210 to 600 nm (extracted wavelength 250 nm, 280 nm); ESI conditions: gas temperature 365° C.; gas flow—8 ml/min; capillary—3.5 kV; nebulizer—40 PSI; fragmentor—145V.

DSC measurements were determined on a TA Instruments Q2000 instrument at a scan rate of 10° C./min, and in a nitrogen atmosphere for all cycles. The sample was scanned from room temperature to 300° C., cooled to −60° C., and reheated to 300° C. The glass transition temperature (T_{g}) was measured on the second heating scan. Data analysis was performed using TA Universal Analysis software. The T_{g} was calculated using an “onset-at-inflection” methodology.

All computations utilized the Gaussian09 program¹. The calculations were performed with the hybrid density functional theory (DFT) method, B3LYP,² and the 6-31G* (5d) basis set.³ The singlet state calculations used the closed shell approximation, and the triplet state calculations used the open shell approximation. All values are quoted in electronvolts (eV). The HOMO and LUMO values were determined from the orbital energies of the optimized geometry of the singlet ground state. The triplet energies were determined as the difference between the total energy of the optimized triplet state and the optimized singlet state. 1. Gaussian 09, Revision A.02, Frisch, M. J. et al.; Gaussian, Inc., Wallingford Conn., 2009.2. (a) Becke, A. D. J. Chem. Phys. 1993, 98, 5648. (b) Lee, C.; Yang, W.; Parr, R. G. Phys. Rev B 1988, 37, 785. (c) Miehlich, B.; Savin, A.; Stoll, H.; Preuss, H. Chem. Phys. Lett. 1989, 157, 200.3. (a) Ditchfield, R.; Hehre, W. J.; Pople, J. A. J. Chem. Phys. 1971, 54, 724. (b) Hehre, W. J.; Ditchfield, R.; Pople, J. A. J. Chem. Phys. 1972, 56, 2257. (c) Gordon, M. S. Chem. Phys. Lett. 1980, 76, 163.

The procedure described in the literature (J. Phys. Chem. A, 2003, 107, 5241-5251) was applied to calculate the reorganization energy (λ⁻) of each molecule which is an indicator of electron mobility.

Some embodiments of the invention will now be described in detail in the following examples.

### Scheme 1: Synthesis of HTL1

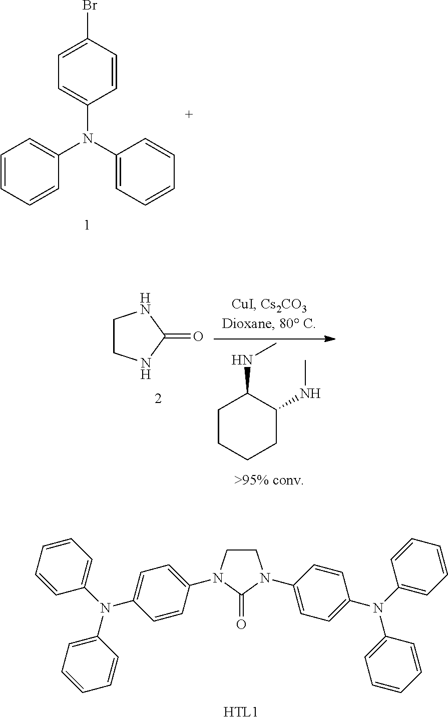

Into a glass jar was weighed 2-imidazolidinone (0.5 g, 6 mmol), 4-bromo-N,N-diphenylaniline (3.8 g, 12 mmol), CuI (110 mg, 0.58 mmol, 10 mol %) and Cs₂CO₃ (3.9 g, 12 mmol, 2 equiv). Nitrogen-spurged dioxane (80 mL) was added, followed by trans-N,N′-dimethylcyclohexane-1,2-diamine-((82 mg, 91 μL, 0.58 mmol, 10 mol %), and the reaction was stirred overnight at 80° C. An aliquot was analyzed by LC-MS, which confirmed approx. 99% conversion to desired product. The solvent was removed under vacuum, and the resulting solid was dissolved in chloroform (150 mL), and washed with water (2×50 mL), brine (3×50 mL), water (2×50 mL). The organic layer was dried with sodium sulfate, filtered, and silica gel was added into the filtrate. The slurry was dried under vacuum, and the free-flowing powder was loaded on to a cartridge, and the crude product was purified by flash column chromatography using a TELEDYNE ISCO purification unit, using an isocratic gradient of 30% chloroform, in hexanes, to give the desired compound (1.5 g) in >99% purity, as determined by LC/MS and 1H-NMR. A small amount of HTL1 was submitted for TGA and DSC analysis for thermal decomposition analysis. A second batch (2 g) was similarly prepared and purified. Both batches were analyzed for purity, and were found to be >99.6% pure by high resolution LC-MS. The batches were combined (3.2 g) and used for device testing. ¹H NMR (400 MHz, CDCl3) δ 7.47 (t, J=10.6 Hz, 4H), 7.32-6.86 (m, 24H), 3.94 (s, 4H); ¹³C NMR (101 MHz, CDCl₃) δ 155.16, 147.80, 143.07, 135.40, 129.14, 125.42, 123.57, 122.33, 119.29, 42.21.

### Experimental for HTL-2

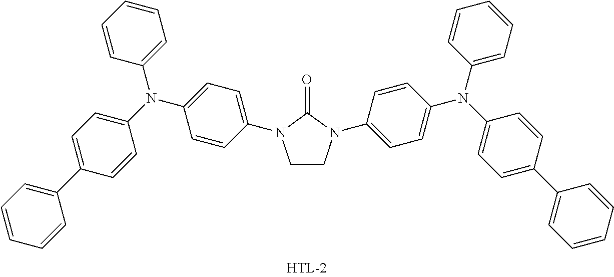 [SMILES: O=C1N(c2ccc(N(c3ccccc3)c3ccc(-c4ccccc4)cc3)cc2)CCN1c1ccc(N(c2ccccc2)c2ccc(-c3ccccc3)cc2)cc1]

In an N₂-purged dry box, 2,5-bis(4-bromophenyl)cyclopentan-1-one (1 g, 2.53 mmol), N-Phenyl-4-biphenylamine (1.55 g, 6.34 mmol), and NaOtBu (0.73 g, 7.61 mmol) were charged into a 250 mL, round bottomed flask, along with toluene (150 mL). To the stirred slurry, tBu₃PPd(crotyl)Cl (50 mg, 0.125 mmol) was added in one portion. The flask was fitted with a Stevens' condenser, and heated to reflux for 18 hours. The reaction was allowed to cool, and was treated with CH₂Cl₂ (150 mL) and water (100 mL). The organic layer was isolated, dried with MgSO₄, and filtered. The solvent was removed, to afford an off white solid. The solid was dry packed onto silica and purified using column chromatography (100 g Biotage column), with a gradient of 0-70% CH₂Cl₂ in hexanes. HTL-2 was isolated as a colorless solid (1.19 g; 64.8% yield). HTL-2 was sublimed to a purity of 99.86% as shown by LC-MS. ¹H NMR (400 MHz, Chloroform-d) δ 7.34 (t, J=1.9 Hz, 1H), 7.32 (t, J=2.1 Hz, 4H), 7.26 (dd, J=6.8, 2.2 Hz, 6H), 7.19 (d, J=2.0 Hz, 1H), 7.17 (d, J=1.7 Hz, 2H), 7.15-7.08 (m, 5H), 7.04 (dd, J=8.7, 7.2 Hz, 4H), 6.94-6.70 (m, 8H), 3.84 (s, 4H); 13C NMR (101 MHz, CDCl3) δ 155.27, 147.87, 144.92, 142.98, 140.52, 139.89, 134.33, 131.93, 129.48, 128.83, 128.72, 128.62, 127.88, 126.79, 125.82, 123.23, 121.35, 120.90, 118.94, 42.30.

### Synthesis of HTL-3

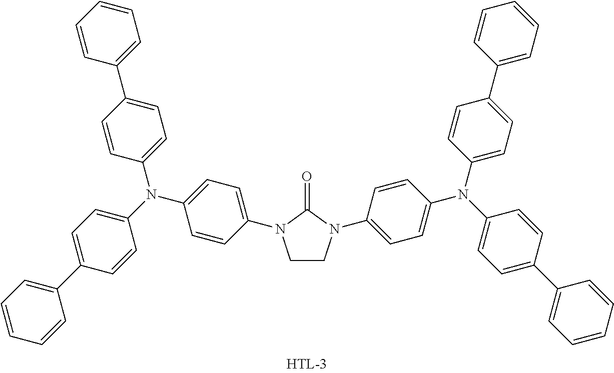 [SMILES: O=C1N(c2ccc(N(c3ccc(-c4ccccc4)cc3)c3ccc(-c4ccccc4)cc3)cc2)CCN1c1ccc(N(c2ccc(-c3ccccc3)cc2)c2ccc(-c3ccccc3)cc2)cc1]

In an N2-purged dry box, 2,5-bis(4-bromophenyl)cyclopentan-1-one (1 g, 2.53 mmol), bis(4-biphenylyl)amine (1.71 g, 5.33 mmol), and NaOtBu (0.73 g, 7.61 mmol) were charged into a 250 mL, round bottomed flask, along with toluene (150 mL). To the stirred slurry, tBu3PPd(crotyl)Cl (41 mg, 0.10 mmol) was added in one portion. The flask was fitted with a Stevens' condenser, and heated to reflux for 18 hours. The reaction was allowed to cool, and was treated with CH₂Cl₂ (1 L) and water (100 mL). A large amount of solvent was used, because of the suspected low solubility of HTL-3. The organic layer was isolated, dried with MgSO₄, and filtered. The solvent was removed, to afford an off white solid. The solid was dry packed onto silica, and purified by column chromatography (100 g Biotage column), with a gradient of 0-20% EtOAc, in hexanes, over 4 column lengths. HTL-3 was isolated as colorless solid (1.29 g; 58.1% yield).

### OLED Device Fabrication and Testing

All organic materials were purified by sublimation before deposition. OLEDs were fabricated onto an ITO coated glass substrate that served as the anode, and topped with an aluminum cathode. All organic layers were thermally deposited by chemical vapor deposition, in a vacuum chamber with a base pressure of <10⁻⁷ torr. The deposition rates of organic layers were maintained at 0.1-0.05 nm/s. The aluminum cathode was deposited at 0.5 nm/s. The active area of the OLED device was “3 mm×3 mm,” as defined by the shadow mask for cathode deposition.

Each cell, containing HILL HIL2, HTL, EML host, EML dopant, ETL, or EIL, was placed inside a vacuum chamber, until it reached 10⁻⁶ torr. To evaporate each material, a controlled current was applied to the cell, containing the material, to raise the temperature of the cell. An adequate temperature was applied to keep the evaporation rate of the materials constant throughout the evaporation process.

For the HIL1 layer, N4,N4′-diphenyl-N4,N4′-bis(9-phenyl-9H-carbazol-3-yl)-[1,1′-biphenyl]-4,4′-diamine was evaporated at a constant 1 A/s rate, until the thickness of the layer reached 800 Angstrom. Simultaneously, the dipyrazino[2,3-f:2′,3′-h]quinoxaline-2,3,6,7,10,11-hexacarbonitrile layer was evaporated at a constant 0.5 A/s rate, until the thickness reached 50 Angstrom. For the HTL layer, N-([1,1′-biphenyl]-4-yl)-9,9-dimethyl-N-(4-(9-phenyl-9H-carbazol-3-yl)phenyl)-9H-fluoren-2-amine was evaporated at a constant 1 A/s rate, until the thickness reached 400 Angstrom (Device 1). For Device 2, HTL-2 was deposited at a constant 1 A/s rate, until the thickness reached 400 Angstrom. For the EML layer, 9-(3-(4,6-diphenyl-1,3,5-triazin-2-yl)phenyl)-9′-phenyl-9H,9′H-3,3′-bicarbazole (GH-1, host) and Iridium, tris[2-(4-methyl-5-phenyl-2-pyridinyl-KN)phenyl-κC]—(GD-1, dopant) were co-evaporated, until the thickness reached 400 Angstrom. The deposition rate for host material was 0.85 A/s, and the deposition for the dopant material was 0.15 A/s, resulting in a 15% doping of the host material. For the ETL layer, 2,4-bis(9,9-dimethyl-9H-fluoren-2-yl)-6-(naphthalen-2-yl)-1,3,5-triazine were co-evaporated with lithium quinolate(Liq), until the thickness reached 300 Angstrom. The evaporation rate for the ETL compounds, and Liq was 0.5 A/s. Finally, “20 Angstrom” of a thin electron injection layer (Liq) was evaporated at a 0.5 A/s rate. See Tables 1 and 2.

The current-voltage-brightness (J-V-L) characterizations for the OLED devices were performed with a source measurement unit (KEITHLY 238) and a luminescence meter (MINOLTA CS-100A). EL spectra of the OLED devices were collected by a calibrated CCD spectrograph.

TABLE 1
Device Materials for Device 1

| | Name |
|---|---|
| Hole Injection | N4,N4′-diphenyl-N4,N4′-bis(9-phenyl-9H-carbazol-3- |
| Material (HIL)1 | yl)-[1,1′-biphenyl]-4,4′-diamine |
| Hole Injection | dipyrazino[2,3-f:2′,3′-h]quinoxaline-2,3,6,7,10,11- |
| Material (HIL2) | hexacarbonitrile |
| Hole Transporting | N-([1,1′-biphenyl]-4-yl)-9,9-dimethyl-N-(4-(9-phenyl- |
| Material(HTL) | 9H-carbazol-3-yl)phenyl)-9H-fluoren-2-amine |
| Ph Green Host | 9-(3-(4,6-diphenyl-1,3,5-triazin-2-yl)phenyl)-9′- |
| (GH-1) | phenyl-9H,9′H-3,3′-bicarbazole |
| Ph Green Dopant | Iridium,tris[2-(4-methyl-5-phenyl-2-pyridinyl- |
| (GD-1) | κN)phenyl-κC]- |
| Ref ETL | 2,4-bis(9,9-dimethyl-9H-fluoren-2-yl)-6-(naphthalen- |
| | 2-yl)-1,3,5-triazine |
| Electron Injection | lithium quinolate |
| Material (EIL) | |

TABLE 2
Device Materials for Device 2

| | Name |
|---|---|
| Hole Injection | N4,N4′-diphenyl-N4,N4′-bis(9-phenyl-9H-carbazol-3- |
| Material (HIL1) | yl)-[1,1′-biphenyl]-4,4′-diamine |
| Hole Injection | dipyrazino[2,3-f:2′,3′-h]quinoxaline-2,3,6,7,10,11- |
| Material (HIL2) | hexacarbonitrile |
| Hole Transporting | HTL-2 |
| Material(HTL) | |
| Ph Green Host | 9-(3-(4,6-diphenyl-1,3,5-triazin-2-yl)phenyl)-9′- |
| (GH-1) | phenyl-9H,9′H-3,3′-bicarbazole |
| Ph Green Dopant | Iridium,tris[2-(4-methyl-5-phenyl-2-pyridinyl- |
| (GD-1) | κN)phenyl-κC]- |
| Ref ETL | 2,4-bis(9,9-dimethyl-9H-fluoren-2-yl)-6-(naphthalen- |
| | 2-yl)-1,3,5-triazine |
| Electron Injection | lithium quinolate |
| Material (EIL) | |

The molecular structures of the molecules that comprise each layer are shown below.

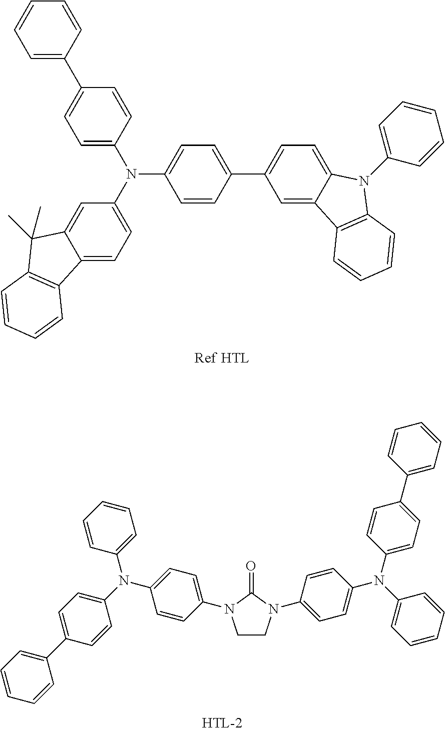 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; O=C1N(c2ccc(N(c3ccccc3)c3ccc(-c4ccccc4)cc3)cc2)CCN1c1ccc(N(c2ccccc2)c2ccc(-c3ccccc3)cc2)cc1]

As seen in the device result (Table 3), the devices containing HTL2 had better (higher) efficiency when compared to the device containing the reference compound HTL.

TABLE 3

| | Efficiency | Efficiency |
|---|---|---|
| | @1000 nit | @10 mA/cm2 |
| | [cd/A] | [cd/A] |
| Device 1 with Ref HTL | 44.4 | 40.8 |
| Device 2 with HTL-2 | 50.0 | 48.4 |

The compounds of the present invention enable a shallowing of the HOMO energy, compared to compounds representative of U.S. Pat. No. 8,022,617. For example, see the compounds below.

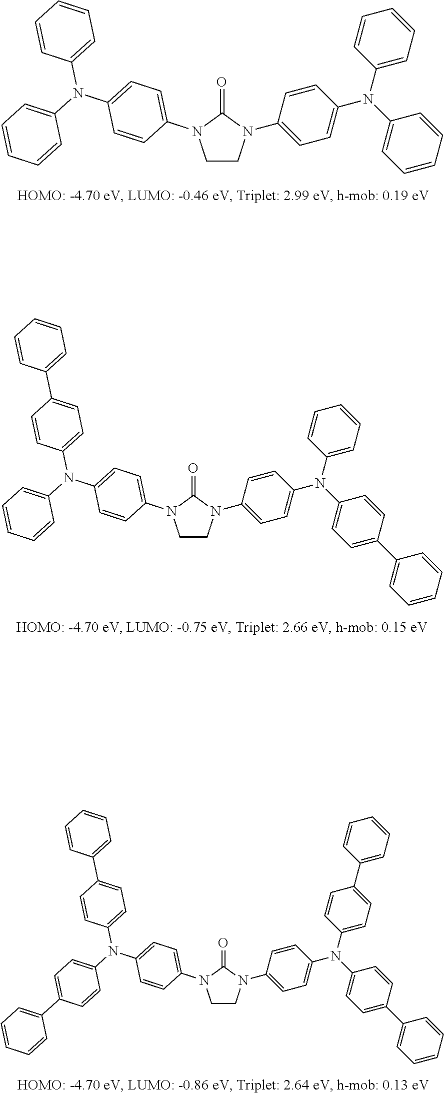 [SMILES: O=C1N(c2ccc(N(c3ccccc3)c3ccccc3)cc2)CCN1c1ccc(N(c2ccccc2)c2ccccc2)cc1 ; O=C1N(c2ccc(N(c3ccccc3)c3ccc(-c4ccccc4)cc3)cc2)CCN1c1ccc(N(c2ccccc2)c2ccc(-c3ccccc3)cc2)cc1 ; O=C1N(c2ccc(N(c3ccc(-c4ccccc4)cc3)c3ccc(-c4ccccc4)cc3)cc2)CCN1c1ccc(N(c2ccc(-c3ccccc3)cc2)c2ccc(-c3ccccc3)cc2)cc1]

The following comparative compounds have calculated HOMO values that are deeper than the ideal range discussed above.

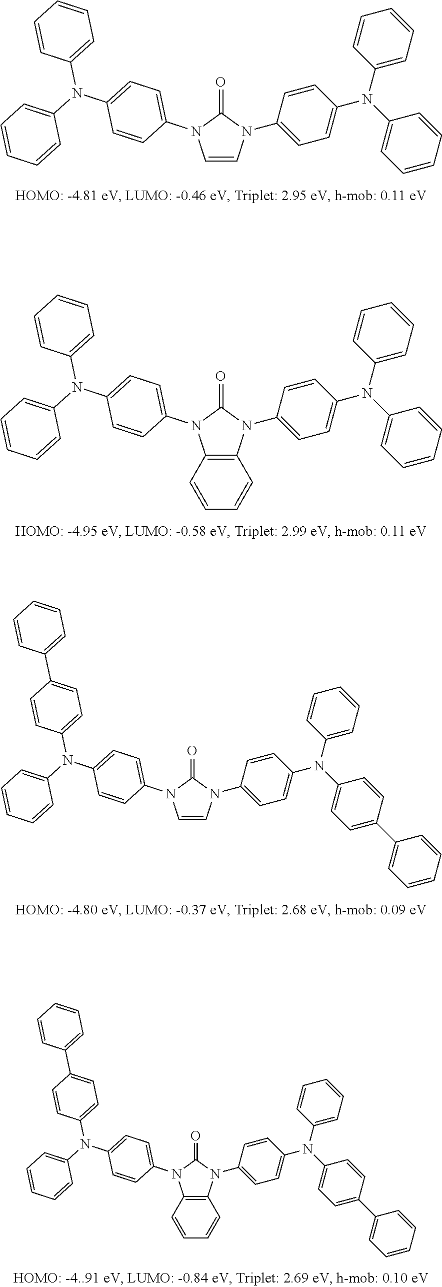 [SMILES: O=c1n(-c2ccc(N(c3ccccc3)c3ccccc3)cc2)ccn1-c1ccc(N(c2ccccc2)c2ccccc2)cc1 ; O=c1n(-c2ccc(N(c3ccccc3)c3ccccc3)cc2)c2ccccc2n1-c1ccc(N(c2ccccc2)c2ccccc2)cc1 ; O=c1n(-c2ccc(N(c3ccccc3)c3ccc(-c4ccccc4)cc3)cc2)ccn1-c1ccc(N(c2ccccc2)c2ccc(-c3ccccc3)cc2)cc1 ; O=c1n(-c2ccc(N(c3ccccc3)c3ccc(-c4ccccc4)cc3)cc2)c2ccccc2n1-c1ccc(N(c2ccccc2)c2ccc(-c3ccccc3)cc2)cc1]

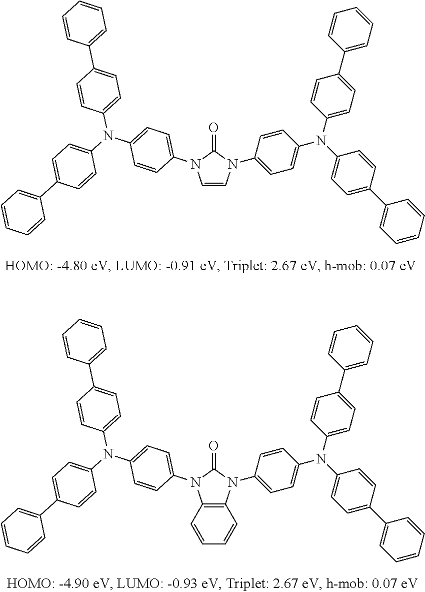 [SMILES: O=c1n(-c2ccc(N(c3ccc(-c4ccccc4)cc3)c3ccc(-c4ccccc4)cc3)cc2)ccn1-c1ccc(N(c2ccc(-c3ccccc3)cc2)c2ccc(-c3ccccc3)cc2)cc1 ; O=c1n(-c2ccc(N(c3ccc(-c4ccccc4)cc3)c3ccc(-c4ccccc4)cc3)cc2)c2ccccc2n1-c1ccc(N(c2ccc(-c3ccccc3)cc2)c2ccc(-c3ccccc3)cc2)cc1]

1. A composition comprising at least one compound of Formula 1 below: 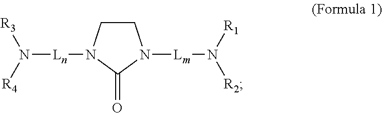
wherein, R1, R2, R3 and R4 are each, independently, selected from the following: an unsubstituted alkyl, a substituted alkyl, an unsubstituted heteroalkyl, a substituted heteroalkyl, an unsubstituted aryl, a substituted aryl, an unsubstituted heteroaryl, or a substituted heteroaryl; and wherein R1 and R2 may be optionally fused to form one or more ring structures; and wherein R3 and R4 may be optionally fused to form one or more ring structures; and
Lₘ and Lₙ are each, independently, selected from the following: an unsubstituted alkylene, a substituted alkylene, an unsubstituted heteroalkylene, a substituted heteroalkylene, an unsubstituted arylene, a substituted arylene, an unsubstituted heteroarylene, or a substituted heteroarylene; and
wherein, for Formula 1, one or more hydrogen atoms may optionally be substituted with deuterium.

2. The composition of claim 1, wherein, for Formula 1, Lₘ and Lₙ each independently, an unsubstituted (3- to 30-membered)heteroarylene, a substituted (3- to 30-membered)heteroarylene, an unsubstituted (C6-C30)arylene, or a substituted (C60C30)arylene.

3. The composition of claim 1, wherein Formula 1, Lₘ and Lₙ each independently, selected from one of the following structures:

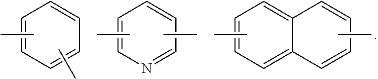

4. The composition of claim 1, wherein, for Formula 1, R₁, R₂, R₃ and R₄, are each, independently, selected from an unsubstituted (C6-C30)arylene, or a substituted (C6-C30)aryl, an unsubstituted (3- to 30-membered)heteroaryl, or a substituted (3- to 30-membered)heteroaryl.

5. The composition of claim 1, wherein, for Formula 1, R₁, R₂, R₃ and R₄, are each, independently, selected from the following A1) to A48): 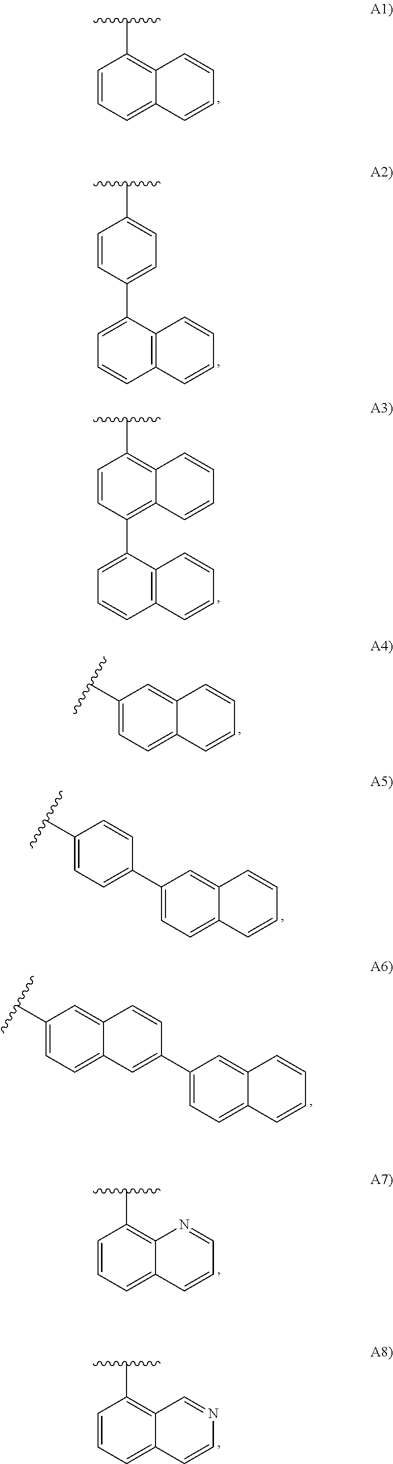 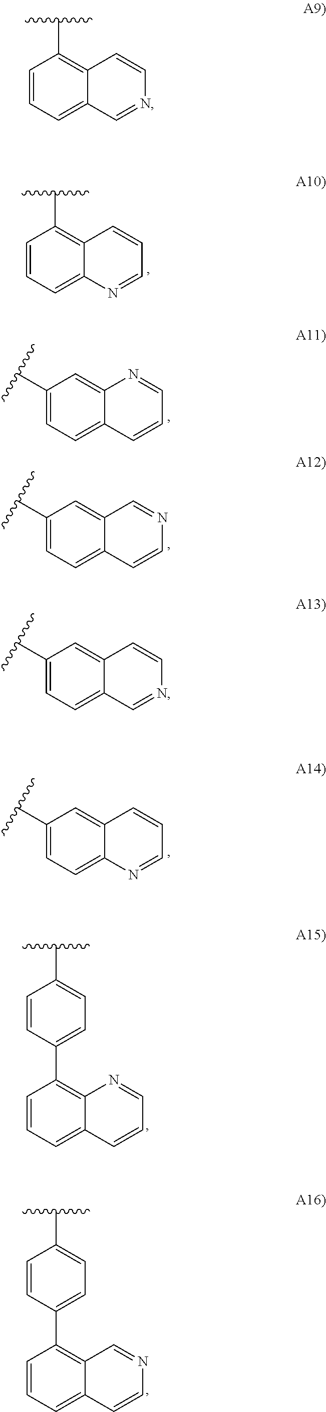 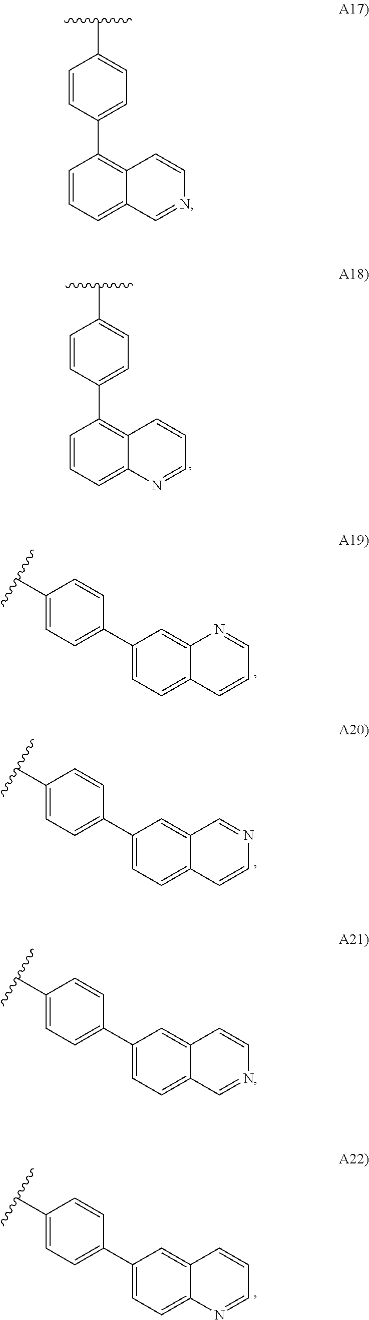 [SMILES: *c1ccc(-c2cccc3cnccc23)cc1 ; *c1ccc(-c2cccc3ncccc23)cc1 ; *c1ccc(-c2ccc3cccnc3c2)cc1 ; *c1ccc(-c2ccc3ccncc3c2)cc1 ; *c1ccc(-c2ccc3cnccc3c2)cc1 ; *c1ccc(-c2ccc3ncccc3c2)cc1] 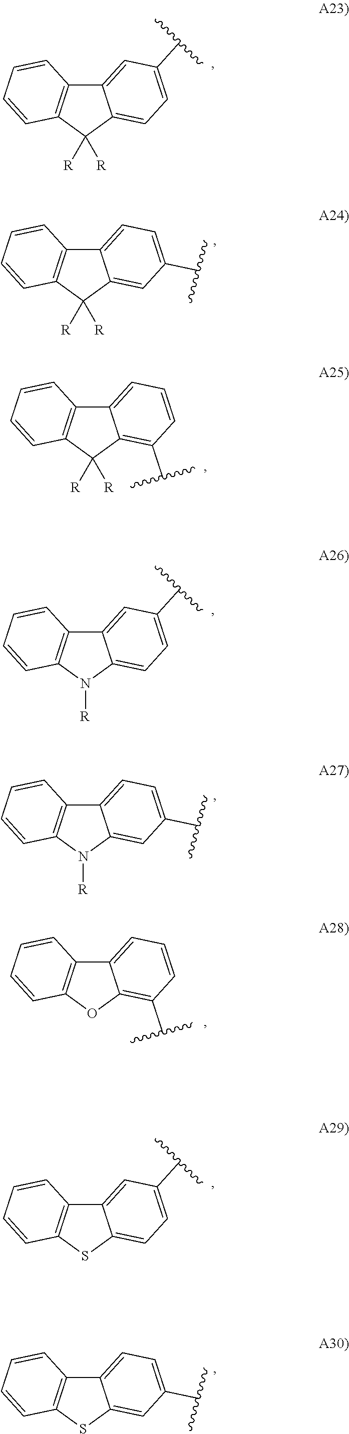 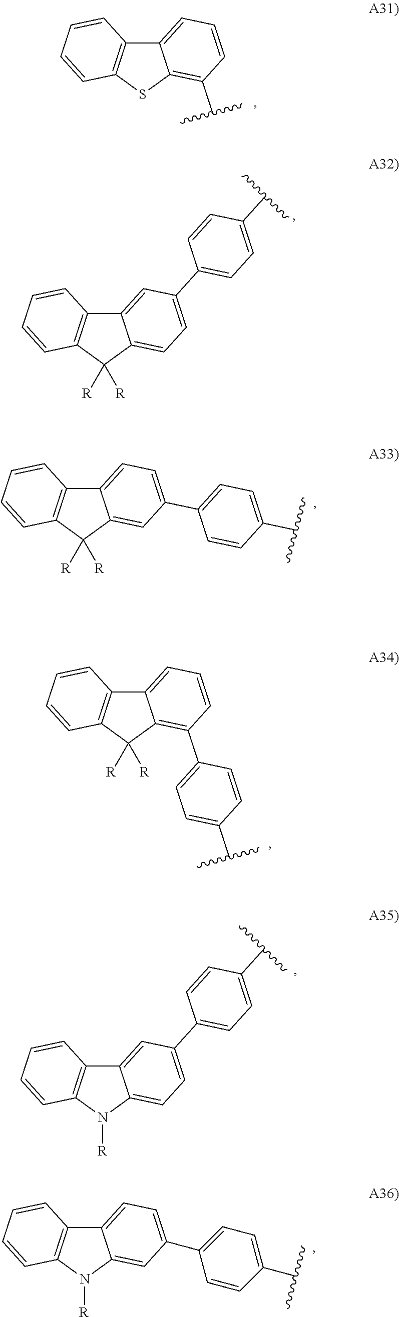 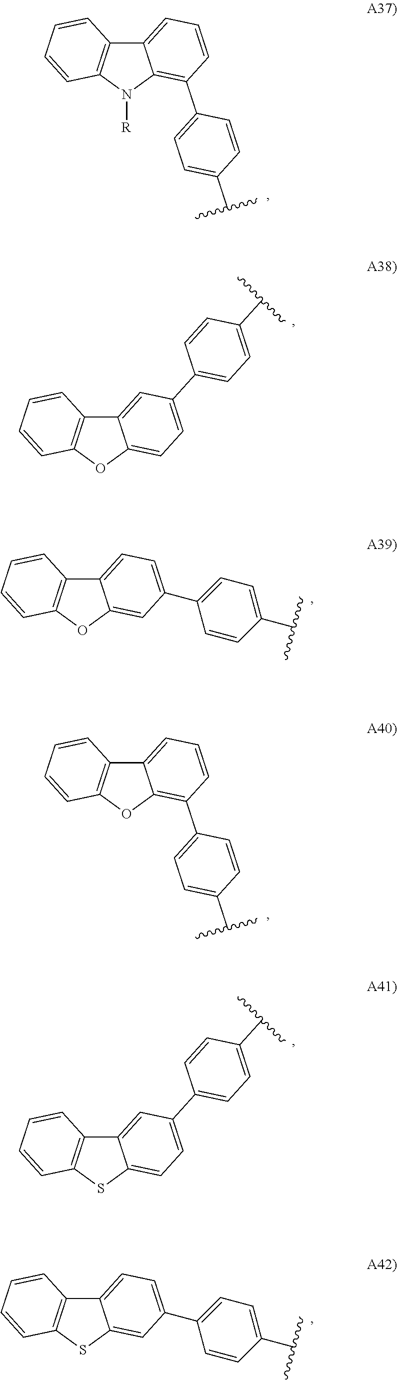 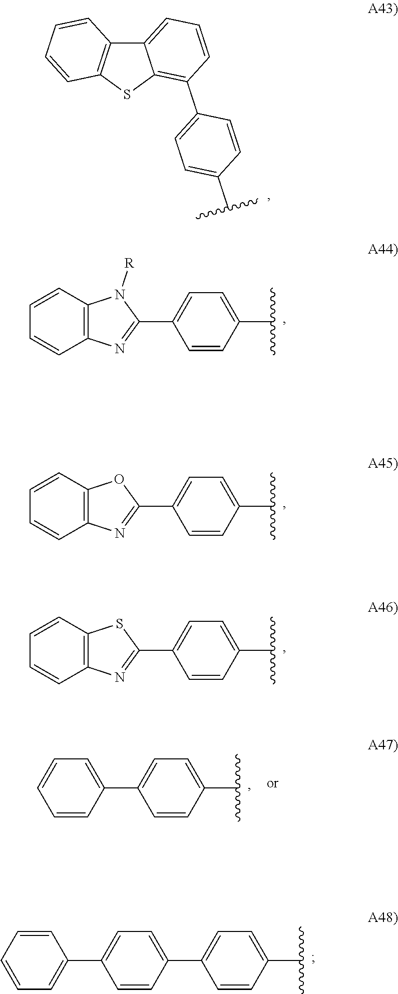
and
wherein for structures A23) through A27), A32) through A37), and A44) each R is independently an alkyl.

6. The composition of claim 1, wherein Formula 1 is selected from Formula 1a:

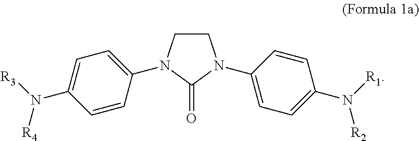

7. The composition of claim 1, wherein the compound of Formula 1 is selected from the following (a) through (o):

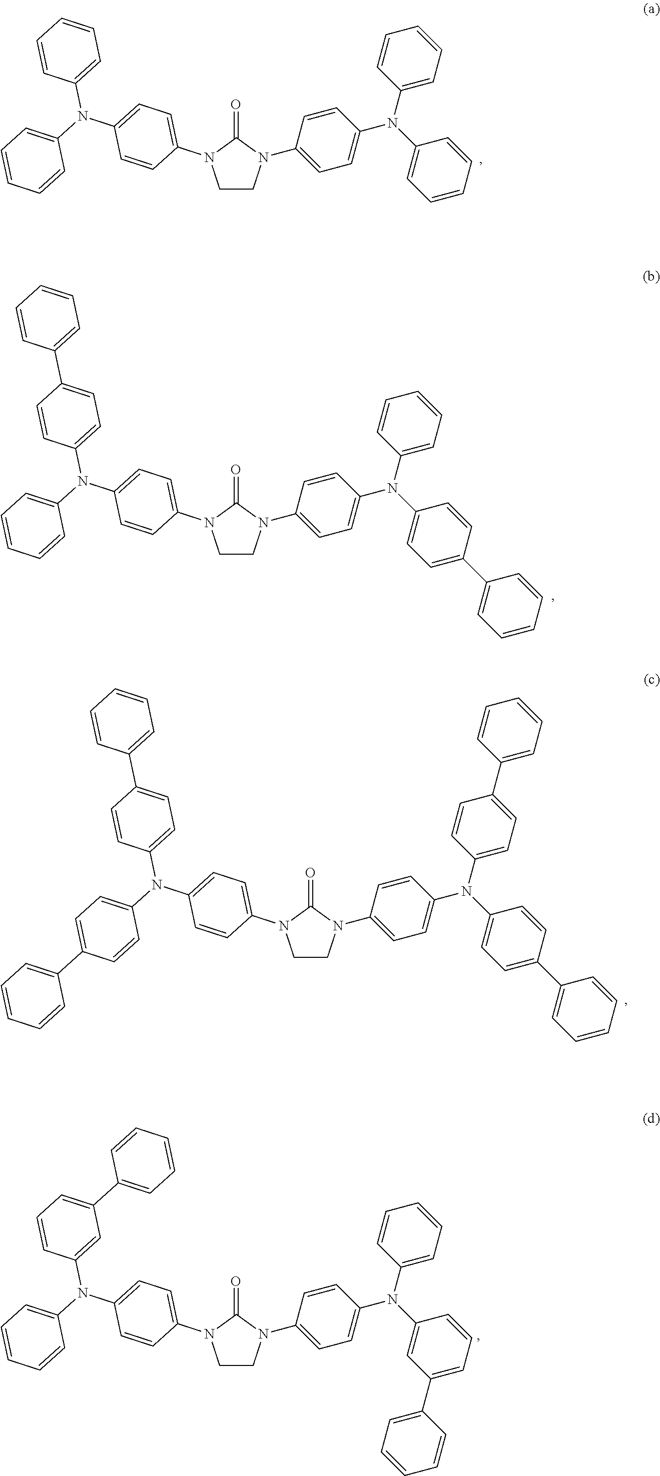 [SMILES: O=C1N(c2ccc(N(c3ccccc3)c3ccccc3)cc2)CCN1c1ccc(N(c2ccccc2)c2ccccc2)cc1 ; O=C1N(c2ccc(N(c3ccccc3)c3ccc(-c4ccccc4)cc3)cc2)CCN1c1ccc(N(c2ccccc2)c2ccc(-c3ccccc3)cc2)cc1 ; O=C1N(c2ccc(N(c3ccc(-c4ccccc4)cc3)c3ccc(-c4ccccc4)cc3)cc2)CCN1c1ccc(N(c2ccc(-c3ccccc3)cc2)c2ccc(-c3ccccc3)cc2)cc1 ; O=C1N(c2ccc(N(c3ccccc3)c3cccc(-c4ccccc4)c3)cc2)CCN1c1ccc(N(c2ccccc2)c2cccc(-c3ccccc3)c2)cc1]

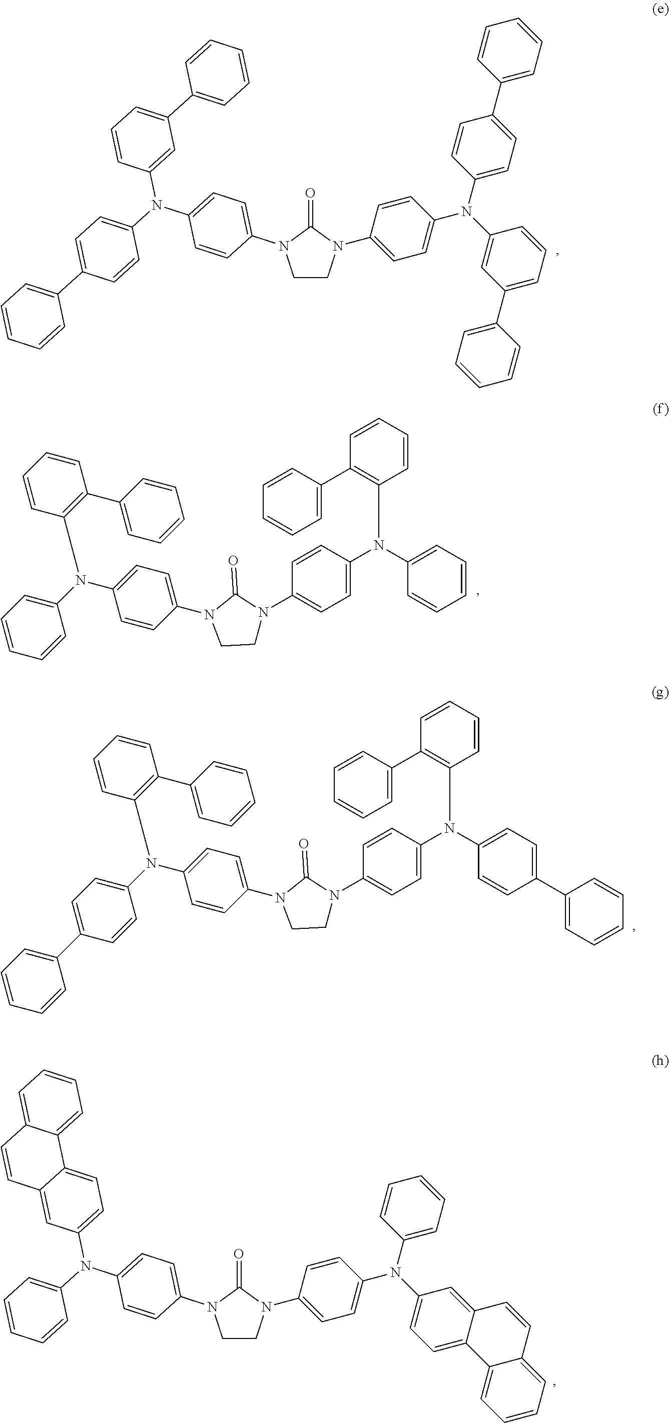 [SMILES: O=C1N(c2ccc(N(c3ccc(-c4ccccc4)cc3)c3cccc(-c4ccccc4)c3)cc2)CCN1c1ccc(N(c2ccc(-c3ccccc3)cc2)c2cccc(-c3ccccc3)c2)cc1 ; O=C1N(c2ccc(N(c3ccccc3)c3ccccc3-c3ccccc3)cc2)CCN1c1ccc(N(c2ccccc2)c2ccccc2-c2ccccc2)cc1 ; O=C1N(c2ccc(N(c3ccc(-c4ccccc4)cc3)c3ccccc3-c3ccccc3)cc2)CCN1c1ccc(N(c2ccc(-c3ccccc3)cc2)c2ccccc2-c2ccccc2)cc1 ; O=C1N(c2ccc(N(c3ccccc3)c3ccc4c(ccc5ccccc54)c3)cc2)CCN1c1ccc(N(c2ccccc2)c2ccc3c(ccc4ccccc43)c2)cc1]

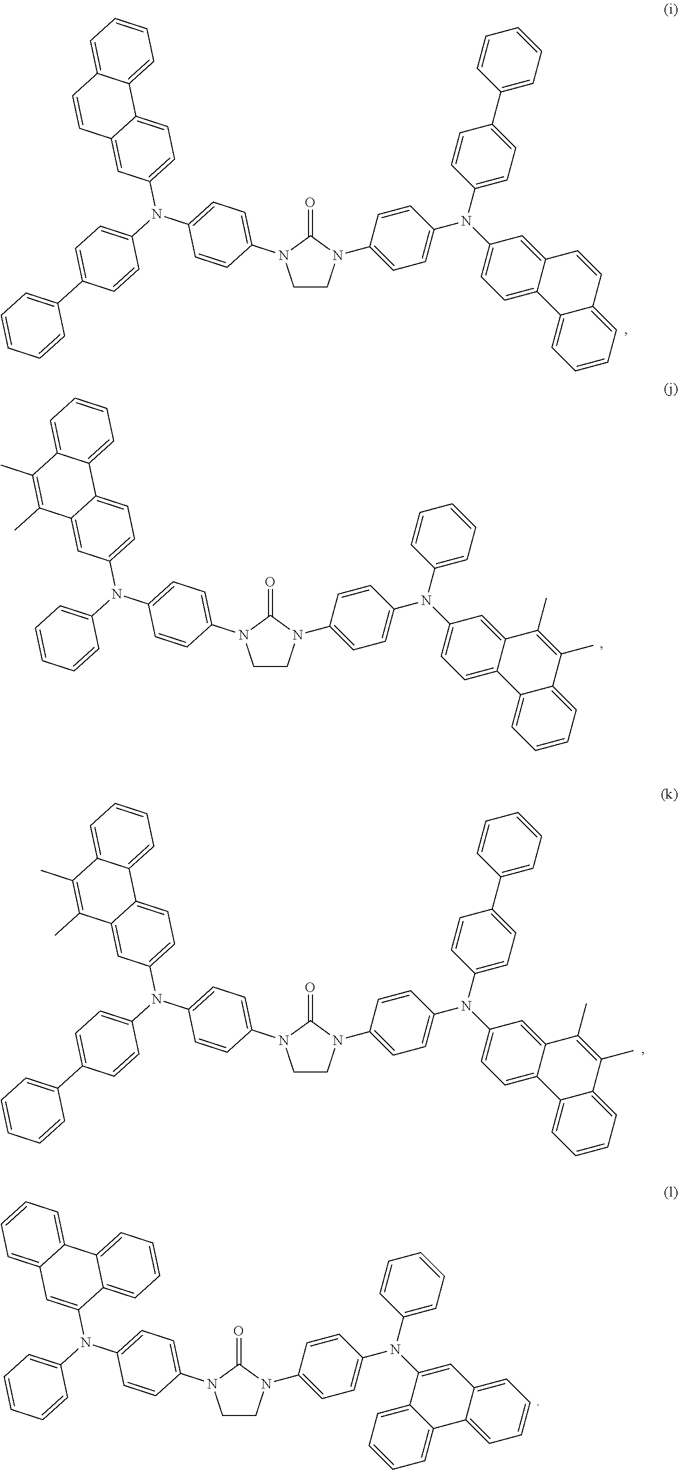

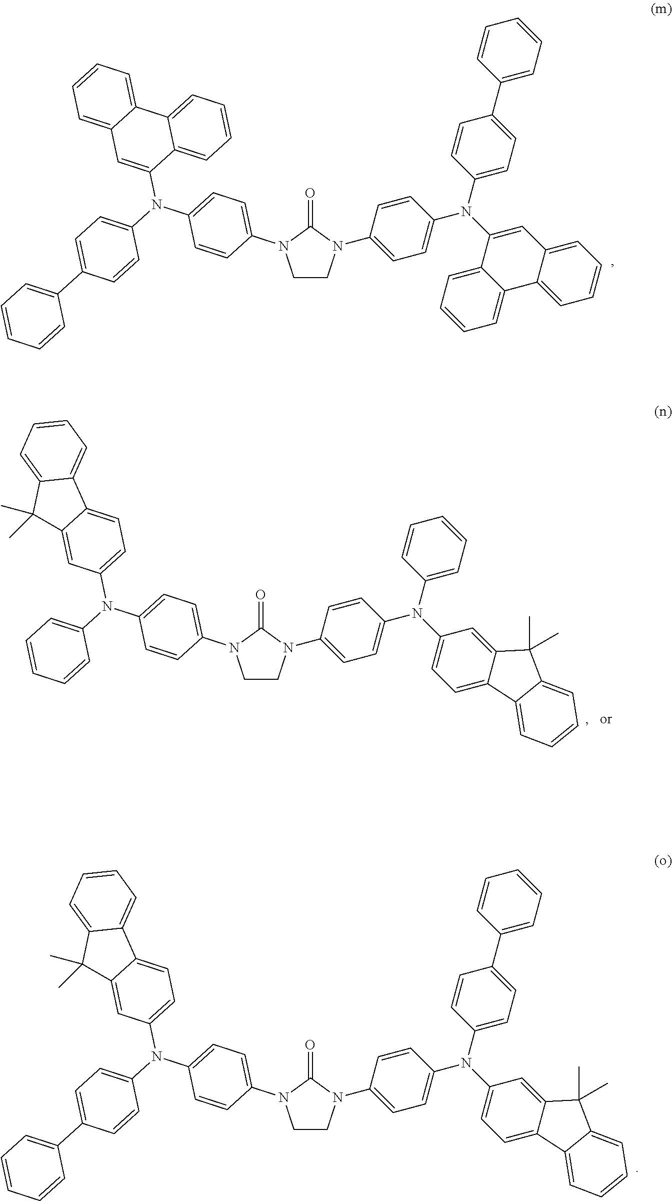 [SMILES: O=C1N(c2ccc(N(c3ccc(-c4ccccc4)cc3)c3cc4ccccc4c4ccccc34)cc2)CCN1c1ccc(N(c2ccc(-c3ccccc3)cc2)c2cc3ccccc3c3ccccc23)cc1 ; CC1(C)c2ccccc2-c2ccc(N(c3ccccc3)c3ccc(N4CCN(c5ccc(N(c6ccccc6)c6ccc7c(c6)C(C)(C)c6ccccc6-7)cc5)C4=O)cc3)cc21 ; CC1(C)c2ccccc2-c2ccc(N(c3ccc(-c4ccccc4)cc3)c3ccc(N4CCN(c5ccc(N(c6ccc(-c7ccccc7)cc6)c6ccc7c(c6)C(C)(C)c6ccccc6-7)cc5)C4=O)cc3)cc21]

8. An article comprising at least one component formed from the composition of claim 1.

9. A film comprising at least one layer formed from the composition of claim 1.

10. An electronic device comprising at least one component formed from the composition of claim 1.
